(19) United States
(12) Patent Application Publication
(10) Pub. No.: US 2026/0335239 A1
(43) Pub. Date: Oct. 29, 2026

(54) SPRAY DRIED EXCIPIENT BLENDS

(71) Applicant: Dow Global Technologies LLC, Midland, MI (US)

(72) Inventors: Luqing Qi, Midland, MI (US); Yifan Wang, Lake Jackson, TX (US); Xue Chen, Pearland, TX (US)

(21) Appl. No.: 19/491,144

(22) PCT Filed: May 29, 2024
(86) PCT No.: PCT/US2024/031353

§ 371 Date: Dec. 8, 2025

Related U.S. Application Data
(60) Provisional application No. 63/579,521, filed on Aug. 30, 2023.

(51) Int. Cl. A61K 9/16; A61K 9/20

(52) U.S. Cl. CPC A61K 9/1641; A61K 9/1611; A61K 9/1623; A61K 9/2009; A61K 9/2018; A61K 9/2031

(57) ABSTRACT

A granular composition containing blended excipients is made by spray-drying sugar alcohol and polyalkylene oxide in the presence of a minor amount of hydrophobic flow aid. The spray-dried granular composition may have superior flowability compared with physically blended compositions. Tablets made from the spray-dried composition may have superior crush-resistance.

### FIELD

This application relates to the field of excipients useful for oral pharmaceuticals.

### INTRODUCTION

Oral pharmaceutical tablets typically contain a small quantity of active ingredient in a larger quantity of excipient. The excipients dilute the active ingredient and may perform a large number of other functions, such as controlling the rate at which the active ingredient is released, helping the active ingredient to be absorbed, moderating side effects, extending shelf life, providing a durable tablet, providing lubrication during tablet pressing, modifying flavor, and providing a distinctive appearance to the tablet. Excipients must also be safe for use in oral medications and must generally be able to meet regulatory and/or approval criteria.

Many materials are approved and used as excipients. Some common examples include cellulose and its derivatives, calcium phosphates, calcium carbonates, calcium sulfates, halites, metal oxides such as titanium dioxide, silicates and their derivatives such as fumed silica and colloidal silica, carbohydrates such as sugars or other sweeteners, starches, fatty alcohols, fatty acid salts, waxes, polyethylene glycol polymers, acrylic polymers and proteins.

It is desirable to identify combinations of approved excipients that can provide improved processability or properties, such as better flowability or better crush resistance for the resulting tablet, without compromising other properties of the excipient.

### SUMMARY

One aspect of this invention is a process to make a granular excipient composition, comprising the steps of
1. making a solution that contains the following dry components:
   a. From 5 to 50 weight percent of a water-soluble polyalkylene glycol that is solid up to at least 35 °C;
   b. From 50 to 95 weight percent of sugar alcohol that is solid up to at least 50 °C, in a solvent, wherein the weight percentages are based on the total weight dry ingredients, excluding the solvent; and
2. Spray-drying the solution in a drying chamber in the presence of hydrophobic flow-aid in a quantity sufficient to reduce adhesion of spray dried particles with surfaces of the drying chamber, to form a dry granular composition,
wherein all percentages are based on the combined weight of polyalkylene glycol and sugar alcohol.

A second aspect of the present invention is a dry granular composition that is made by the process of this invention and that contains:
a. From 5 to 50 weight percent of a water-soluble polyalkylene glycol that is solid up to at least 35 °C; and
b. From 50 to 95 weight percent of sugar alcohol that is solid up to at least 50 °C; and
c. hydrophobic flow aid, wherein all percentages are based on the combined weight of polyalkylene glycol and sugar alcohol.

A third aspect of this invention is a granular composition comprising:
a. From 5 to 50 weight percent of a water-soluble polyalkylene glycol that is solid up to at least 35 °C; and
b. From 50 to 97 weight percent of a sugar alcohol that is solid up to at least 50 °C; and
c. From 0.1 to 5 weight percent hydrophobic flow aid,
   wherein individual particles of the granular composition contain a mixture containing the polyalkylene glycol and sugar alcohol and the hydrophobic flow aid is predominantly on the surface of the particles, and wherein all percentages are based on the combined weight of polyalkylene glycol and sugar alcohol.

A fourth aspect of this invention is the method to use a granular composition of this invention, comprising the step of compressing the granular composition into a solid tablet.

A fifth aspect of this invention is a solid tablet comprising: (1) a pharmaceutically effective amount of an oral pharmaceutical dispersed in (2) a compressed granular composition of this invention.

The granular compositions of this invention are useful as excipients for oral pharmaceuticals and other tableted supplements, vitamins, actives, etc. In some embodiments, the granular compositions of this invention may have high flowability, and tablets made using the granular composition may have high crush resistance. The granular compositions may also be useful to modify the dissolution speed of the resulting tablets.

### DETAILED DESCRIPTION

Granular compositions of this invention contain polyalkylene glycol, sugar alcohols and hydrophobic flow aid. Sugar alcohols, polyalkylene glycols and some hydrophobic flow aids, such as fumed silica, are common all excipients that are approved for use in oral pharmaceuticals and are widely available.

Polyalkylene glycols contain repeating units that meet Formula 1:

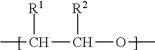

wherein each of R¹ and R² is hydrogen or an alkyl group. In some embodiments, each of R¹ and R² independently contains no more than 6 carbon atoms or no more than 4 carbon atoms or no more than 2 carbon atoms or no more than 1 carbon atom. In some embodiments, R¹ and R² together contain on average no more than 8 carbon atoms or no more than 6 carbon atoms or no more than 4 carbon atoms or no more than 2 carbon atoms or no more than 1 carbon atom or no more than 0.5 carbon atoms or no more than 0.25 carbon atoms. In some embodiments, R¹ and R² are both hydrogen in at least 80 percent of repeating units, or at least 90 percent or essentially 100 percent. In some embodiments, the polyoxyalkylene chains contain, as desired, ethylene oxide [—CH₂—CH₂—O—] units, propylene oxide [—CH₂(CH₃)—CH₂—O—] units or both types of units in any desired ratio in the same molecular chain.

In some embodiments, the polyalkylene glycol is polyethylene glycol. In some embodiments, the polyalkylene glycol is a polyethylene glycol/polypropylene glycol copolymer. In some embodiments, the polyalkylene glycol is a poloxamer, which is a triblock copolymer composed of a central chain of polypropylene glycol flanked by two chains of polyethylene glycol. In many poloxamers, the polypropylene glycol chain is hydrophobic, and the polyethylene glycol chains are hydrophilic. Examples of polyethylene glycols used as excipients are described in Rowe et al, Handbook of Pharmaceutical Excipients, 5^{th} Ed at 545-550 (2006).

The polyalkylene glycol should be water soluble. In some embodiments, it is soluble in water at 20 °C up to at least 20 weight percent, or at least 40 weight percent or at least 50 weight percent or at least 60 weight percent. No maximum solubility is desired, but solubility over 100 weight percent or 80 weight percent may be unnecessary for some uses.

The polyalkylene glycol should be solid up to 35 °C. In some embodiments, it has a melting temperature of at least 40 °C or at least 45 °C or at least 50 °C or at least 54 °C. In some embodiments, it has a melting temperature of no more than 100 °C or no more than 80 °C or no more than 70 °C or no more than 65 °C.

In some embodiments, the polyalkylene glycol has a number average molecular weight of at least 2000 Da or at least 3000 Da or at least 4000 Da or at least 5000 Da or at least 6000 Da or at least 7000 Da or at least 7500 Da or at least 8000 Da. In some embodiments, the polyalkylene glycol has a number average molecular weight of at most 25,000 Da or at most 20,000 Da or at most 15,000 Da or at most 12,000 Da or at most 10,000 Da.

In some embodiments, the polyalkylene glycol has a viscosity at 100 °C of at least 200 cSt or at least 400 cSt or at least 450 cSt or at least 500 cSt. In some embodiments, the polyalkylene glycol has a viscosity at 100 °C of at most 5000 cSt or at most 3000 cSt or at most 2000 cSt or at most 1000 cSt.

Suitable polyalkylene glycols are commercially available under the CARBOWAX SENTRY™ trademark and the Kollisolv trademark. Others can be made by known processes, such as polymerization of corresponding alkylene oxide monomers in the presence of an acid or base initiator. See for example, US Patent Publication US 2007/0179199 A1 and “Introduction of Polyethylene Glycol (PEG)”, made available by BOC Sciences at https://peg.bosci.com/resources/technical-information/introduction-of-polyethylene-glycol-peg.

Sugar alcohols are alkyl polyols having a hydroxyl group attached to each carbon atom. In some embodiments, the sugar alcohol is linear, meeting formula 2:

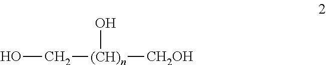

wherein n is a number of repeating units. In some embodiments, the sugar alcohol is cyclic.

In some embodiments, the sugar alcohol contains at least 4 carbon atoms (n=2) or 5 carbon atoms (n=3) or 6 carbon atoms (n=4). In some embodiments, the sugar alcohol contains at most 18 carbon atoms (n=16) or 12 carbon atoms (n=10) or 8 carbon atoms (n=6) or 6 carbon atoms (n=4).

The sugar alcohol should be solid up to 50 °C. In some embodiments, it has a melting temperature of at least 70 °C or at least 90 °C or at least 100 °C. In some embodiments, it has a melting temperature of no more than 220 °C or no more than 205 °C or no more than 180 °C or no more than 170 °C or no more than 165 °C.

Examples of suitable sugar alcohols include erythritol, xylitol, mannitol and sorbitol. In some embodiments, the sugar alcohol is mannitol. In some embodiments, the sugar alcohol is sorbitol. In some embodiments, the sugar alcohol comprises a mixture of two or more sugar alcohols. Mixtures may have a lower melting temperature than pure sugar alcohols, allowing the melting temperature to be controlled by controlling the mixture. For example, mannitol melts at about 165 °C, and sorbitol melts at about 200 °C, whereas eutectic mixtures of mannitol and sorbitol can melt at temperatures below 100 °C depending on the proportions selected.

Appropriate sugar alcohols are commercially available. Others can be made by hydrogenation of the corresponding sugar.

The granular composition contains 5 to 50 weight percent polyalkylene glycol, based on the combined weight of polyalkylene glycol and sugar alcohol. In some embodiments, the granular composition contains at least 6 weight percent polyalkylene glycol or at least 7 weight percent or at least 8 weight percent or at least 9 weight percent or at least 10 weight percent, based on the combined weight of polyalkylene glycol and sugar alcohol. In some embodiments, the granular composition contains at most 40 weight percent polyalkylene glycol or at most 30 weight percent or at most 25 weight percent or at most 20 weight percent or at most 18 weight percent or at most 16 weight percent, based on the combined weight of polyalkylene glycol and sugar alcohol.

The granular composition contains from 50 to 95 weight percent sugar alcohol, based on the combined weight of polyalkylene glycol and sugar alcohol. In some embodiments, the granular composition contains at least 60 weight percent sugar alcohol or at least 70 weight percent or at least 75 weight percent or at least 80 weight percent or at least 82 weight percent or at least 84 weight percent or at least 88 weight percent, based on the combined weight of polyalkylene glycol and sugar alcohol. In some embodiments, the granular composition contains at most 92 weight percent sugar alcohol, or at most 91 weight percent or at most 90 weight percent, based on the combined weight of polyalkylene glycol and sugar alcohol.

The granular composition further contains a hydrophobic flow aid that is suitable for pharmaceutical use. In some embodiments, the flow aid comprises silica, fumed silica, an aluminosilicate or a mixture thereof. In some embodiments, the flow aid comprises hydrophobic fumed silica, and in some embodiments the flow aid consists essentially of hydrophobic fumed silica. Fumed silica is a powder containing fused amorphous silica with low bulk density and high surface area. Fumed silica is hydrophilic, but can be rendered hydrophobic by treating with a hydrophobic material such as silicone.

In some embodiments, the hydrophobic flow aid has a D90 particle size (90% of particles smaller than) at most 200 mesh (74 micron) or at most 270 mesh (53 micron) or at most 325 mesh (44 micron). In some embodiments, the hydrophobic flow aid has a D90 particle size (90% of particles smaller than) at least 500 mesh (25 micron) or at most 400 mesh (37 micron).

In some embodiments, the hydrophobic flow aid has a BET surface area of at least 100 m²/g or at least 150 m²/g or at least 175 m²/g. In some embodiments, the hydrophobic flow aid has a BET surface area of at most 500 m²/g or at most 300 m²/g or at most 250 m²/g or at most 225 m²/g.

In some embodiments, the hydrophobic flow aid has a loose bulk density of at most 200 g/L or at most or at most 150 g/L or at most 100 g/L or at most 70 g/L r at most 60 g/L. In some embodiments, the hydrophobic flow aid has a loose bulk density of at least 10 g/L or at least 20 g/L or at least 40 g/L or at least 50 g/L.

The quantity of hydrophobic flow aid used in the process is sufficient to reduce adhesion of spray dried particles with surfaces of the drying chamber. In some embodiments, the granular composition contains at least 0.1 weight percent hydrophobic flow aid or at least 0.3 weight percent or at least 0.5 weight percent or at least 0.7 weight percent or at least 0.9 weight percent or at least 1 weight percent, based on the combined weight of polyalkylene glycol and sugar alcohol. In some embodiments, the granular composition contains at most 5 weight percent hydrophobic flow aid or at most 4 weight percent or at most 3 weight percent or at most 2 weight percent, based on the combined weight of polyalkylene glycol and sugar alcohol.

Hydrophobic flow aids, such as hydrophobic fumed silica, are known and commercially available, such as under the DOWSIL™, Cabosil and Aerosil trademarks. Fumed silica can also be made by flame pyrolysis of silicon tetrachloride or by from vaporizing quartz sand in a high temperature electric arc, and can be converted to hydrophobic fumed silica by treatment with organosilane. See, for example, European Patent Publication EP0928818A2.

The granular composition optionally contains from 0 to 22 weight percent of other excipients useful for oral pharmaceuticals and tablets. A wide range of excipients are available that serve a number of different purposes, such as
Fillers & Diluents,
Binders,
Anti-Caking Agents
Suspension & Viscosity Agents,
Coatings,
Flavoring Agents and Sweeteners,
Disintegrants,
Colorants,
Lubricants & Glidants,
Preservatives,
Surfactants,
Controlled release agents,
Blending and Mixing Aids; and
Compression Aids.

Examples of useful excipients include microcrystalline cellulose, dibasic phosphates such as calcium phosphates, calcium carbonates, calcium sulfates, halites, metal oxides such as titanium dioxide, colloidal silica, carbohydrates such as sugars or other sweeteners, starches, cellulose ethers, microcrystalline cellulose, fatty alcohols, fatty acid salts, waxes, acrylic polymers and proteins. More common examples include magnesium stearate, calcium phosphate, starch, silicone/titanium dioxide, colloidal silicon dioxide, microcrystalline cellulose, stearic acid, sodium starch glycolate, gelatin, talc, sucrose, calcium stearate, edible dyes, croscarmellose, hydroxypropyl cellulose, hydroxypropyl methyl cellulose, ethyl cellulose, povidone and crospovidone. Some excipients are further described in Kibbe, Handbook of Pharmaceutical Excipients, Third Ed. (2000); PCT Publication WO 2011/024028 from Paragraphs [9] to [19], inclusive and PCT Publication WO 2021/231946 A1, Paragraphs to inclusive.

In some embodiments, the granular composition contains at least 1 weight percent other excipients or at least 2 weight percent or at least 3 weight percent or at least 4 weight percent or at least 5 weight percent, based on the combined weight of sugar alcohol and polyalkylene glycol. In some embodiments, the granular composition contains at most 20 weight percent other excipients or at most 18 weight percent or at most 16 weight percent or at most 14 weight percent or at most 12 weight percent or at most 10 weight percent or at most 8 weight percent or at most 6 weight percent, based on the combined weight of sugar alcohol and polyalkylene glycol.

In some embodiments, the granular composition further contains one or more active ingredients. Active ingredients can include any pharmaceutical, probiotic or nutrient that is solid at room temperature and is suitable for oral administration. Examples of active ingredients are described in numerous sources

such as PCT Publication WO 2021/231946 A1, Paragraphs to inclusive. Examples of potential active ingredients include vitamins such as Vitamins A, B, C, D and/or E and other nutritional supplements; probiotics such as bacteria; pain relievers such as aspirin, acetaminophen or ibuprofen; decongestants; antibiotics; antacids; and other pharmaceuticals. In some embodiments, the granular composition contains a single active ingredient. In some embodiments, such as multivitamins, the composition may contain multiple active ingredients.

The active ingredient may be present in a pharmaceutically-effective concentration, which may vary depending on the active ingredients. Some pharmaceutical tablets may contain active doses as low as 5 mg or lower. On the other hand, some vitamin tablets may contain 1000 mg of active vitamins or more.

In the process of this invention, the sugar alcohol and polyalkylene glycol and optionally other excipients and/or active ingredients (collectively called the “dry components”) are mixed in solvent to form a solution. Then the solution is spray-dried in the presence of the hydrophobic flow aid to make the granular composition of the invention.

For clarity, the sugar alcohol and the polyalkylene glycol must be mixed into the solution, and the hydrophobic flow aid must be introduced separately into the spray-drying chamber during the spray-drying of the solution. However, any other excipients and active ingredients may be added to the granular composition in one or more stages of its production and use. The other excipients and/or active ingredients may be dissolved or suspended in the solution before the granular composition is spray dried. They may be introduced into the spray-drying chamber when the granular compositions are dried. They may be physically mixed with the granular composition after it is recovered from the spray-drying and/or before it is compressed into tablets.

To form the solution, the sugar alcohol and the polyalkylene glycol and optionally other dry components are mixed in solvent until a homogeneous solution is formed. In some embodiments, the solvent comprises water or comprises at least 50 weight percent water (called aqueous solvent). In some embodiments, the solvent comprises lower alkanol, dichloromethane, chloroform or acetonitrile. In some embodiments, the solvent consists essentially of water. In some embodiments, excess solvent is kept low, in order to minimize the need for drying in the spray drying step.

In some embodiments, the solution contains at least 1 weight percent dry components or at least 5 weight percent or at least 10 weight percent. In some embodiments, the solution contains at most 60 weight percent dry components or at most 50 weight percent. In some embodiments, the solution contains at least 40 weight percent solvent, or at least 50 weight percent. In some embodiments, the solution contains at most 99 weight percent solvent, or at most 95 weight percent or at most 90 weight percent. In some embodiments, one or more of the dry components may already be dissolved or suspended in solvent, and this solvent may be considered when calculating the total solvent in the solution. The ratio of dry components in the solution reflects the proportions already discussed.

In some embodiments, the dry components of the solution may impact the acidity of the solution. In some embodiments, the pH of the solution is at least 4 or at least 5 or at least 6 or at least 7. In some embodiments, the pH of the solution is at most 10 or at most 8 or at most 7.5 or at most 7. In some embodiments, pH may be controlled by buffers. Suitable buffers are known and commercially-available. Examples may include citrates and citrate salts and phosphate salts.

The solution is spray-dried according to known techniques. Spray-drying equipment is commercially available with instructions for its use, and spray-drying is described in many publications such as: Santos et al., “Spray Drying-A Overview”, available at: http://dx.doi.org/10.5772/intechopen.72247; “Spray Dry Manual” published by Bete Performance Spray Engineering at www.BETE.com; and More Swati et al, “Review on Spray Drying Technology” 4 (2) IJPCBS 219-225 (2014).

In summary, the spray-drying step has the following sub-steps:
1. The solution is atomized (sprayed) to form atomized droplets in a drying chamber, and simultaneously the hydrophobic flow aid is introduced into the drying chamber in a quantity sufficient to reduce adhesion of spray dried particles with surfaces of the drying chamber;
2. The atomized droplets are contacted in the drying chamber with a heated gas under conditions that dry the droplets into dry granules in the presence of the hydrophobic flow aid; and
3. The dried granules are separated from the gas and recovered.

In some embodiments, the drying chamber may have a cylindrical section and a narrowing conic section at the bottom where dried granules are collected and removed from the drying chamber. Several different types of atomizers are known, including spray nozzles and rotary atomizers. In some embodiments the heated gas is air, and in some embodiments the heated gas is an inert gas such as nitrogen or carbon dioxide.

Spray drying processes are sometimes classified as:
1. Co-current: Both the solution and the drying gas are fed into the drying chamber at the top of the chamber and flow together in the same direction out of the bottom of the chamber.
2. Counter-current: The solution is fed into the drying chamber from the top to middle of the chamber and flows to the bottom and out from the bottom. The drying gas is fed into the drying chamber near or below the bottom of the cylindrical section of the chamber and flows up and out of the top of the drying chamber, in the opposite direction from the solution/granules.
3. Mixed-Mode: The solution is fed into the chamber and atomized near the bottom of the cylindrical section of the chamber. The drying gas is fed into the drying chamber at the top of the chamber. Dried granules fall to the bottom of the chamber and are recovered there, while the drying gas may flow out at the bottom of the chamber or at an intermediate point.
   The spray-drying step in the present invention may use any one of these configurations. In some embodiments it is mixed-mode spray drying.

The hydrophobic flow aid is sprayed into the drying chamber separately from the solution. For example, in an embodiment of a mixed-mode spray-dryer, the solution containing sugar alcohol and polyalkylene oxide may be fed and atomized at the usual point near the bottom of the cylindrical section of the chamber, and the hydrophobic flow aid may be sprayed separately into the drying chamber near or above the top of the cylindrical section.

In some embodiments, other excipients and/or active ingredients may be also sprayed into the drying chamber separately from the solution as described for the hydrophobic flow aid.

The spray-drying process of this invention is carried out at a temperature and a rate of gas flow at which the atomized droplets dry rapidly without substantially degrading the ingredients of the granular composition. Typically, the temperature at the inlet is higher than at the outflow. In some embodiments, the inlet temperature is at least 100 °C or at least 120 °C or at least 130 °C or at least 140 °C. In some embodiments the inlet temperature is no more than 200 °C or no more than 180 °C or no more than 160 °C or no more than 150 °C. Gas flow rates will vary depending on the equipment; in general, the rate should be fast enough to rapidly dry the atomized particles without agglomerating them but slow enough that the granules are not carried out with the gas.

In some embodiments, the atomized droplets become sufficiently dry to prevent substantial further agglomeration in no more than 60 seconds or 30 seconds or 15 seconds. There is no minimum drying time, but in some embodiments drying times of less than 1 second or 5 seconds are unnecessary. In some embodiments, the resulting granules contain no more than 15 weight percent water or no more than 10 weight percent or no more than 8 weight percent or no more than 7 weight percent. In some embodiments, the resulting granules contain at least 1 weight percent water or at least 2 weight percent or at least 3 weight percent or at least 5 weight percent.

The spray-dried granules are separated from the drying gas and recovered by known means, such as by cyclone separators, or bag filters or electrostatic precipitators.

We hypothesize that spray-drying causes the granular composition to have a different morphology than it would have if the components were physically blended. Physical blending would produce a mixture containing discrete particles of sugar alcohol and of polyalkylene oxide and of other excipients and/or active ingredients, with little mixing within the particles. On the other hand, we hypothesize that spray drying the solution makes particles that each contain a mixture of the sugar alcohol and polyalkylene oxide (and any other components dissolved or suspended in the solution). In some embodiments, the mixture of sugar alcohol and polyalkylene oxide in the particles may be substantially homogeneous. When the spray-drying is performed in the presence of separately added hydrophobic flow aid, we hypothesize that the flow aid is predominantly embedded at or near the surface of the particles.

The ratio of dry components in the granules reflects the proportions already discussed.

In some embodiments, particles of the granular composition have a mean particle size (Dmean) of at least 5 micron or at least 8 micron or at least 10 micron or at least 20 micron or at least 50 micron or at least 75 micron or at least 100 micron. In some embodiments, particles of the granular composition have a mean particle size of at most 300 micron or at most 250 micron or at most 200 micron or at most 150 micron or at most 100 micron or at most 75 micron or at most 50 micron or at most 40 micron or at most 30 micron. For example, some large particle granular compositions may have a mean particle size from 75 to 200 micron or from 100 to 150 micron, whereas some small particle granular compositions may have a mean particle size from 5 to 50 micron or from 10 to 30 micron.

In some embodiments, the angle of repose for a large particle granular composition of this invention is no more than 40° or no more than 38° or no more than 36° or no more than 34° or no more than 32° or no more than 30°. In some embodiments, the angle of repose for a large particle granular composition of this invention is at least 20° or at least 25° or at least 26° or at least 27° or at least 28°. In some embodiments, the angle of repose for a large particle granular composition of this invention is no more than 50° or no more than 40° or no more than 38°. In some embodiments, the angle of repose for a small particle granular composition of this invention is at least 25° or at least 30° or at least 33°. In some embodiments, the angle of repose for the granular compositions of this invention is at least 1° lower than the angle of repose for a physical blend of the starting ingredients (having a similar particle size profile) or at least 2° lower or at least 3° lower or at least 5° lower or at least 7° lower. In some embodiments, the angle of repose for the granular compositions of this invention is no more than 15° lower than the angle of repose for a physical blend of the starting ingredients (having a similar particle size profile) or no more than 10° lower. In some embodiments, low angle of repose may indicate that the granular composition flows well and is easy to process.

The granular compositions of this invention may be compressed into tablets by known means. Before compression, the granular composition may optionally be blended with active ingredients or other excipients, as previously described. The blending may be carried out using known equipment, such as impellers or rotating drums. In some embodiments, the composition to be compressed contains at least 50 weight percent granular composition of this invention (as recovered from the spray-drying), or at least 60 weight percent, or at least 70 weight percent or at least 80 weight percent or at least 90 weight percent or at least 95 weight percent or at least 98 weight percent. In some embodiments, the composition to be compressed contains up to 100 weight percent granular composition of this invention (as recovered from the spray-drying), or no more than 99.99 weight percent or no more than 99.9 weight percent or no more than 99.5 weight percent or no more than 99 weight percent.

Equipment to compress tablets is commercially available, with instructions for its use. Optimum compression pressure to make tablets varies widely depending on components selected for the granular composition. In some embodiments, the compression takes place at a pressure of at least 10 MPa or at least 20 MPa or at least 40 MPa or at least 50 MPa. In some embodiments, the compression takes place at a pressure of no more than 500 MPa or no more than 400 MPa or no more than 300 MPa or no more than 250 MPa. In some embodiments, the compression takes place at a temperature of at least 0 °C or at least 20 °C In some embodiments, the compression takes place at a temperature of no more than 70 °C or no more than 40 °C.

The resulting tablet may be any size that is appropriate for oral administration. In some embodiments, the sum of length plus width plus depth for the tablet is at least 9 mm or at least 11 mm or at least 13 mm. In some embodiments, the sum of length plus width plus depth for the tablet is at most 35 mm or at most 30 mm or at most 25 mm. Optionally, the tablet may be coated after it is compressed, such as with gelatin or a delayed-release coating.

In some embodiments, tablets of this invention may have a crush resistance (hardness) of at least 3500 g/mm or at least 4000 g/mm or at least 5000 g/mm or at least 6000 g/mm or at least 7000 g/mm or at least 8000 g/mm. There is no maximum desired crush resistance, but in some embodiments crush resistance above 15,000 g/mm or 10,000 g/mm is unnecessary. In some embodiments, tablets of this invention may have a crush resistance (hardness) that is at least 1000 g/mm higher than tablets made from a physically blended mixture of the same ingredients or at least 2000 g/mm higher. There is no maximum desired crush resistance improvement, but in some embodiments improvement above 8000 g/mm or 6000 g/mm is unnecessary.

In some embodiments, tablets of this invention may dissolve more slowly than tablets made from a physically blended mixture of the same ingredients.

### TEST METHODS

Properties described in this paper are measured using the following test methods, unless it is clear from the context that a different method is intended.

| Melting Temperature | Vendor provides |
|---|---|
| Melting Temperature of | According to method of European Pharmacopeia. |
| Polyalkylene Oxide | |
| Viscosity of | According to the method of European Pharmacopeia. |
| Polyalkylene Oxide | |
| Particle Size | Measured using a Beckman Coulter LS 13 320 laser diffraction particle |
| | size analyzer equipped with a Tornado Dry Powder system. Laser light at |
| | 780 nm is used to size the particles with diameters ranging from 0.4 to 2000 |
| | μm by light diffraction. The relative amplitude of each pattern is used to |
| | compute the relative volume of spherical particles of that size, based on the |
| | Fraunhofer and Mie models of light scattering by particles. |
| Surface area | Brunauer-Emmett-Teller (BET) test |
| Bulk Density of | ASTM D7481-18 |
| Powder | |
| Angle of Repose | measured by ASTM C1444 |
| Hardness of tablets | Measured using a TA.XT Plus 1000 texture analyzer made by Stable Micro |
| | Systems. Tablet is secured on the instrument platform. The analyzer is |
| | programmed to lower the crusher at a steady speed and crush tablet. The |
| | sensor records maximum force when tablet gets crushed. Hardness is |
| | defined by the unit force, which is maximum force divided by tablet |
| | thickness. |
| Dissolution Time of | Dissolution time is the time interval between when tablets are dropped into |
| Tablet | water and when tablet components are fully dispersed in the water. The |
| | tablet is dropped into an individual container with 200 mL deionized water |
| | at room temperature and stirred with a stir bar at 20 rpm until fully |
| | dissolved. |

### EXAMPLES

The following Examples illustrate some embodiments of the invention.

The Examples use the materials in Table 1.

TABLE 1

| | | Chemical Description, Chemical | |
|---|---|---|---|
| Ingredient Type | Product Name | formula, or Structure | Source |
| Sugar Alcohol | D-Mannitol | C₆H₁₄O₆ | Sigma |
| Polyalkylene | Polyethylene glycol | H(OCH₂CH₂)ₙOH, Mw 8,000, | TDCC |
| Glycol | | powder | |
| Inorganic | Silicon dioxide | colloidal, highly dispersed, | Sigma |
| Excipient | | EMPROVE ® ESSENTIAL, Ph. | |
| | | Eur., JP, NF, E 551 | |
| Flow aid | CAB-O-SIL ® TS-720 | hydrophobic fumed silica | Cabot |
| | fumed silica | | |

TDCC = The Dow Chemical Company

For Inventive Examples 1 to 5 (IE1-IE5), the sugar alcohol, polyalkylene glycol and inorganic excipient from Table 1 are blended with water in the proportions shown in Table 2 to form a homogeneous solution containing about 20 weight percent solids. (The inorganic excipient does not dissolve in the solution but forms a suspension.) The slurries are spray dried according to the following procedure, to make a granular composition. The spray drier is a Mobile Minor spray dryer (GEA Process Engineering Inc.) fitted with a two-fluid nozzle atomizer. The spray drying is performed under an inert atmosphere of nitrogen. Nitrogen is supplied to the atomizer at ambient temperature at 1 bar and 50% flow, which is equivalent to 6.0 kg/hour of flow rate. The solution is fed into the atomizer at about 30 mL/min using a peristaltic pump (Masterflex L/S). Heated nitrogen is fed as drying gas into the top of the drying chamber at a flow rate of about 20 SCFM. The inlet temperature is set at 140 °C, and the outlet temperature is equilibrated at 40-50 °C by fine tuning the solution feed rate. Flow aid is added at the top of the drying chamber through a Coperion K-TRON screw feeder at a feed rate of 0.1 g/min. The resulting spray-dried granular composition is recovered in a cyclone and subsequently vacuum dried at room temperature to removed residual moisture.

For Comparative Example CE1, the same procedure as the Inventive Examples is followed without flow-aid; product adheres to the spray-dryer walls and no product is recovered. For Comparative Example CE3, the same procedure as the Inventive Examples is followed without alkylene glycol. For Comparative Examples CE2 and CE4-CE6, the materials are physically blended until a homogeneously mixed granular composition is obtained. Comparative Examples are shown in Table 2.

The particle sizes and flowability (angle of repose) of each granular composition are measured as described in the Test Methods. Particle sizes are shown in Table 2. Flowability and mixability results are shown in Table 3.

Each granular composition is compressed to make tablets using a Carver compression molder. A 2 gram sample of each granular composition is weighed into the template and a weight of 5000 pounds is applied to make a round-disk shape tablet with a diameter of 13/16 inch. The hardness and dissolution of the tablets are tested as set out in the Test Methods. Results are set out in Table 3. For Dissolution Rate:
very fast=less than 10 seconds,
fast=10 to 60 seconds,
slow=60 to 600 seconds, and
very slow=more than 600 seconds

TABLE 2

| | | Dₘₑₐₙ Particle | D50 particle | Angle of | Hardness | Dissolution |
|---|---|---|---|---|---|---|
| Sample | Formulation | size (micron) | size (micron) | Repose (°) | (g/mm) | Rate * |
| IE1 | Mannitol (90%), PEG 8000 (10%) | 10-30 | 10-30 | 36.0 ± 1.0 | 8000 ± 200 | Very Slow |
| | (spray dried with flow aid) | | | | | |
| IE2 | Mannitol (90%), PEG 8000 (10%), | 100-150 | 100-150 | 28.7 ± 1.0 | — | |
| | (spray dried with flow aid) | | | | | |
| | sieved to 75-425 microns | | | | | |
| IE3 | Mannitol (35%), PEG 8000 (15%) | 10-30 | 10-30 | 34.1 ± 0.5 | 5300 ± 500 | Słow |
| | (spray dried with flow aid) | | | | | |
| IE4 | Mannitol (85%), PEG 8000 (10%), Di-Basic Calcium | 10-30 | 10-30 | 38.7 ± 23 | 4000 ± 120 | Moderate |
| | Phosphate (5%) (spray dried with flow aid) | | | | | |
| IE5 | Mannitol (85%), PEG 8000 (10%), Colloidal silica | 10-30 | 10-30 | 38.6 | 3900 ± 500 | Slow |
| | (5%) (spray dried with flow aid) | | | | | |
| CE1 | Mannitol (90%), PEG 8000 (10%) | N/A* | N/A* | — | — | — |
| | (spray dried without flow aid) | | | | | |
| CE2 | Mannitol (90%), PEG 8000 (10%) (physical blend) | 100-150 | 100-150 | 41.3 ± 1.0 | 1000 ± 200 | Fast |
| CE3 | Mannitol (100%) (spray dried with flow aid) | 10-30 | 10-30 | — | 2300 ± 200 | Moderate |
| CE4 | Mannitol (85%), PEG 8000 (15%) (physical blend) | 100-150 | 100-150 | 38.5 ± 1.4 | 1000 ± 100 | Moderate |
| CE5 | Mannitol (85%), PEG 8000 (10%), Di-Basic Calcium | 100-150 | 100-150 | 37.5 ± 2.1 | 830 ± 100 | Fast |
| | Phosphate (5%) (physical blend) | | | | | |
| CE6 | Mannitol (85%), PEG 8000 (10%), | 100-150 | 100-150 | 37 | 220 ± 20 | Very Slow |
| | Colloidal silica (5%) (physical blend) | | | | | |

A: Fast <5 min, Moderate 5-30 min., Slow 30-60 min, Very Slow >60 mon.
* No Product recovered due to adhesion on the spray-dryer walls.

1. A granular composition comprising:
a) From 5 to 50 weight percent of a water-soluble polyalkylene glycol that is solid up to at least 35 °C; and
b) From 50 to 97 weight percent of a sugar alcohol that is solid up to at least 50 °C; and
c) From 0.5 to 5 weight percent hydrophobic flow aid,
wherein individual particles of the granular composition contain a mixture containing the polyalkylene glycol and sugar alcohol and the hydrophobic flow aid is predominantly on the surface of the particles, and wherein all percentages are based on the combined weight of polyalkylene glycol and sugar alcohol.

2. The granular composition of claim 1 wherein the sugar alcohol contains erythritol, xylitol, mannitol or sorbitol.

3. The granular composition of claim 2 wherein the granular composition contains from 80 to 95 weight percent sugar alcohol, based on the combined weight of polyalkylene glycol and sugar alcohol.

4. The granular composition of claim 3 wherein the sugar alcohol contains mannitol.

5. The granular composition of claim 1 wherein the polyalkylene glycol is a polyethylene glycol or a polyethylene glycol-polypropylene glycol copolymer.

6. The granular composition of claim 5 wherein the polyalkylene glycol has a molecular weight of at least 4000 Da and at most 12,000 Da.

7. The granular composition of claim 6 wherein the granular composition contains from 5 to 15 weight percent polyalkylene glycol, based on the combined weight of polyalkylene glycol and sugar alcohol.

8. The granular composition of claim 1 wherein the hydrophobic flow aid comprises hydrophobic fumed silica.

9. The granular composition of claim 8 wherein the granular composition contains from 0.5 to 3 weight percent hydrophobic fumed silica, based on the combined weight of polyalkylene glycol and sugar alcohol.

10. The granular composition of claim 1 wherein the granular composition contains:
a) from 80 to 95 weight percent erythritol, xylitol, mannitol or sorbitol,
b) from 5 to 15 weight percent polyethylene glycol or a polyethylene glycol-polypropylene glycol copolymer that has a molecular weight of at least 4000 Da and at most 12,000 Da, and
c) from 0.5 to 3 weight percent hydrophobic flow aid that comprises hydrophobic fumed silica,
wherein all percentages are based on the combined weight of polyalkylene glycol and sugar alcohol.

11. The granular composition of claim 10 which further contains from 1 to 20 weight percent of other excipients, based on the combined weight of polyalkylene glycol and sugar alcohol, and wherein the other excipients are selected from the group consisting of dibasic phosphates, calcium carbonates, calcium sulfates, halites, metal oxides, colloidal silica, carbohydrates, starches, cellulose ethers, microcrystalline cellulose, fatty alcohols, fatty acid salts, waxes, acrylic polymers and proteins.

12. The granular composition of claim 10 which has a mean particle size from 5 micron to 50 micron and an angle of repose of no more than 40°.

13. The granular composition of claim 10 which has a mean particle size from 75 micron to 200 micron and an angle of repose of no more than 30°.

14. A solid tablet comprising: (1) a pharmaceutically effective amount of an oral pharmaceutical dispersed in (2) a compressed granular composition of claim 1.

15. A process to make a granular excipient composition comprising the steps of:
a) making a solution that contains the following dry components:
i) From 5 to 50 weight percent of a water-soluble polyalkylene glycol that is solid up to at least 35 °C;
ii) From 50 to 95 weight percent of sugar alcohol that is solid up to at least 50 °C, in a solvent, wherein the weight percentages are based on the total weight dry ingredients, excluding the solvent; and
b) Spray-drying the solution in a drying chamber in the presence of hydrophobic flow aid in a quantity sufficient to reduce adhesion of spray dried particles with surfaces of the drying chamber, to form a dry granular composition,
wherein all percentages are based on the combined weight of polyalkylene glycol and sugar alcohol.
